(19) United States
(12) Patent Application Publication
(10) Pub. No.: US 2009/0076333 A1
(43) Pub. Date: Mar. 19, 2009

(54) Flip Control Retractor Support

(75) Inventor: Todd M. Bjork, River Falls, WI (US)

Correspondence Address: SHEWCHUK IP SERVICES, 3356 SHERMAN CT. STE. 102, EAGAN, MN, 55121, US

(73) Assignee: LEVAHN INTELLECTUAL PROPERTY HOLDING COMPANY, LLC, St. Paul, MN (US)

(21) Appl. No.: 11/856,713

(22) Filed: Sep. 17, 2007

(51) Int. Cl. A61B 1/32

(52) U.S. Cl. 600/210; 600/213

(57) ABSTRACT

A flip control support connection in a surgical retractor system includes a receptacle head having a through-hole for receiving a nipple on a retractor blade in either of two directions. In one direction, flats on the receptacle head mate with flats on the nipple so the blade is rotationally fixed relative to the receptacle head and its handle. The handle/receptacle head can be hand-detached from the nipple/blade, flipped to the opposing orientation, and hand-reattached to provide a second mode of tissue retraction. In the second mode of tissue retraction, the flats on the receptacle head do not mate with the flats on the nipple, and the nipple/blade is free to pivot during retraction relative to the receptacle head/handle.

### CROSS-REFERENCE TO RELATED APPLICATION(S)

None.

### BACKGROUND OF THE INVENTION

The present invention relates to the field of surgical tools, and particularly to the design and manufacture of surgical retractor systems. Surgical retractor systems have long been used during surgery to bias and hold tissue in a desired position. Some retractor systems are bed or post mounted or otherwise mechanically supported relative to the surgical site, while other retractor systems are hand held. In both cases, one or more “blades” make contact with patient tissue being retracted.

In many retractor systems, clamps are used which have a loosened position in which the retractor blade and/or portions of the assembly can be easily moved and a tightened position in which the clamp connection is held rigid. The specific configuration in which the system is assembled is usually determined based upon the location and direction of surgical retraction force desired relative to the surgical site.

Some surgical retractor systems have blades and/or rakes which are hand removable and interchangeable relative to the support structure. Types and sizes of blades used in an operation, and during different stages of one operation, can depend upon a number of factors, including but not limited to the type of the operation, the particular part of the anatomy operated on, the body weight of the individual operated on, and so forth. The surgeon can select which type, width, depth or other shape of retractor blade(s) to use for a particular surgical application from a collection of blades, and thereby have the blade better suited for the desired retraction. It is convenient to be able to change the specific blades mounted to the positioning apparatus during the course of the operation. At the same time, the positioning apparatus for the retractor blades should present minimum obstruction to the surgical team's activities, particularly in the area of an incision.

One common way to connect removable blades to a support structure is by utilizing a nipple fixed to the blade which is removably received in a receptacle head of the retractor support structure. Examples of such removable/changeable blades using nipple connectors are shown in U.S. Pat. Nos. 4,930,932, 5,882,298, 5,902,233, 5,993,385, 6,042,540 and 6,945,933, and in U.S. patent application Ser. No. 11/033,982, each incorporated by reference. Nipple attachments are particularly beneficial for the ability to pivot the retractor blade about the nipple axis.

Surgical retractor systems must be robust and strong, as even a possibility of failure during use is not tolerated. Surgical retractor assemblies should be readily reusable, including sterilizable, for use in multiple surgeries. Surgical retractor systems should maintain a relatively low cost. Improvements in surgical retractor systems can be made in keeping with these goals.

### BRIEF SUMMARY OF THE INVENTION

The present invention is a flip control support connection in a surgical retraction system. The connection is located between a component which contacts tissue of a patient and a handle or similar component which supports the retraction force. The connection has a first attachment position which permits a first mode of tissue retraction. If and/or when the surgeon desires to switch to or use a second mode of tissue retraction, the connection can be hand-separated, the handle component flipped to a new circumferential position, and then the connection quickly hand-reassembled. The surgeon thus has control over which mode of tissue retraction will be used merely by flipping of the handle to the desired orientation for hand assembly. In one aspect, the two modes of tissue retraction control whether the tissue contacting component can freely pivot relative to the handle component, or whether the tissue contacting component is rotationally locked relative to the handle component.

### BRIEF DESCRIPTION OF THE DRAWINGS

FIG. 1 is a manufacturing assembly perspective view of a surgical retractor flip control connection in accordance with the present invention.

FIG. 2 is a plan view of one side of the receptacle head of the flip control connection of FIG. 1.

FIG. 3 is a cross-sectional view of the receptacle head taken along lines 3-3 of FIGS. 2 and 4.

FIG. 4 is a plan view of the other side of the receptacle head of FIGS. 1-3.

FIG. 5 is an end view of the receptacle head of FIGS. 1-4.

FIG. 6 is an enlarged perspective view of the bottom side of the nipple of the flip control connection of FIG. 1.

FIG. 7 is a side view of the nipple of FIG. 6.

FIG. 8 is a bottom view of the nipple of FIGS. 6 and 7.

FIG. 9 is a cross-sectional view of the assembled receptacle head and detent of FIG. 1.

FIG. 10 is a manufacturing assembly side view of a retractor blade and nipple for use with the receptacle head of FIGS. 1-5 and 9.

FIG. 11 is a cross-sectional view of the flip control connection assembled on its “blade secured” side, taken along lines 11-11 in FIG. 12.

FIG. 12 is a cross-sectional view of the assembled flip control connection, taken along lines 12-12 in FIG. 11.

FIG. 13 is a cross-sectional view of the flip control connection assembled on its other “blade free” side, taken along lines 13-13 in FIG. 14.

FIG. 14 is a cross-sectional view of the assembled flip control connection, taken along lines 14-14 in FIG. 13.

While the above-identified drawing figures set forth one or more preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

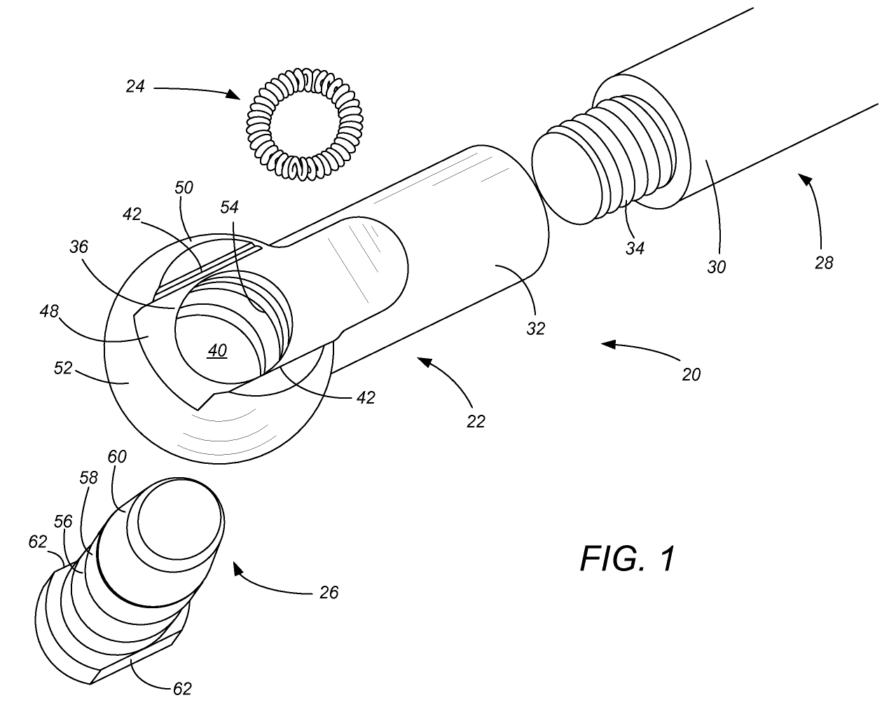

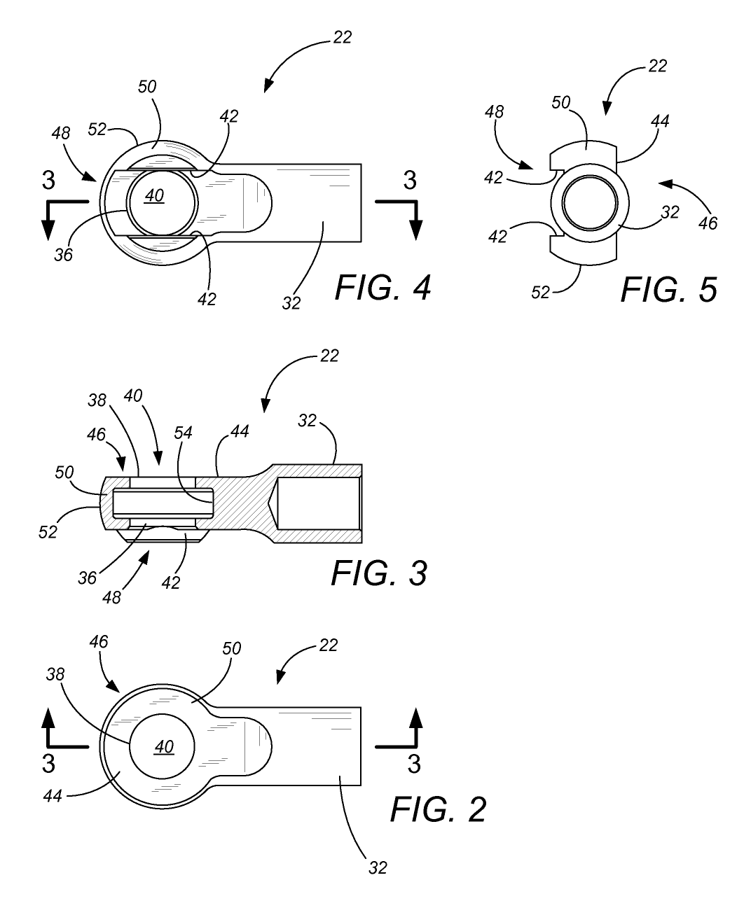

A surgical retractor flip control connection 20 in accordance with the present invention can be accomplished with the embodiment depicted in FIG. 1, which includes a receptacle head 22, a detent mechanism 24 for permanent assembly and use with the receptacle head 22, and a nipple 26. A portion of a handle 28 is shown, with a distal end 30 which can be permanently attached into a proximal end 32 of the receptacle head 22. The handle portion 28 could be hand held, or could alternatively be a distal end of a bar on a table-, post- or bed-mounted retractor system.

Forming the receptacle head 22 separately from the handle portion 28 makes machining of the receptacle head 22 easier and less costly, so the receptacle head 22 can be assembled as a manufacturing step onto any number of different handle structures as known in the surgical retractor art. As another example different than the cylindrical handle 28 shown, the handle portion could alternatively include a toothed shaft used in Codman/Bookwalter retraction systems. The handle portion 28 is depicted in FIG. 1 with helical threads 34 on its distal end 30 for screwing into the proximal end 32 of the receptacle head 22, but many other equivalent attachment methods between the handle 28 and the receptacle head 22 could alternatively or additionally be used, such as press-fitting, soldering, welding, adhesive bonding, etc.

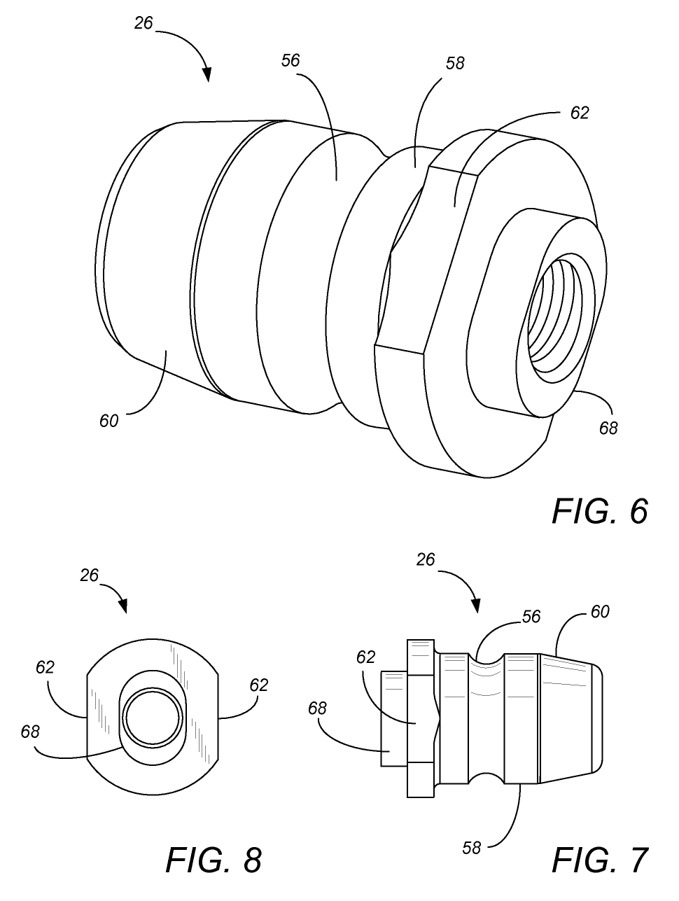

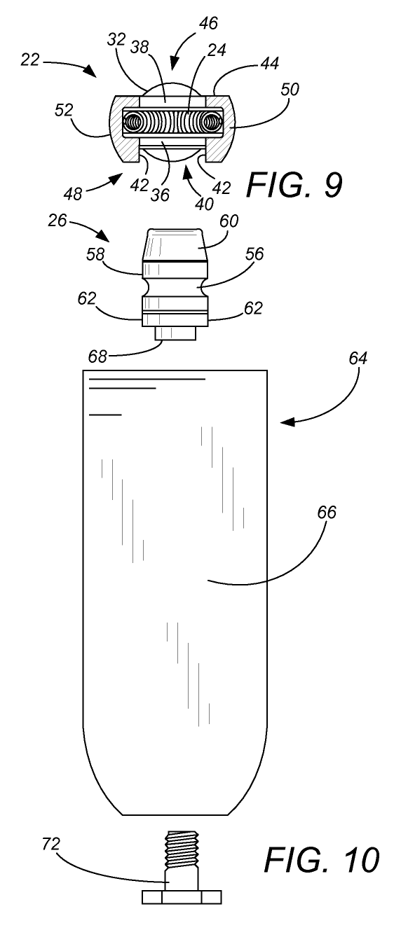

The receptacle head 22 of the present invention includes at least two recesses 36, 38 for the nipple 26. In the preferred embodiment, the two recesses 36, 38 are provided as opposing sides of a single through-hole 40 in the receptacle head 22. One of the recesses 36 has a feature that is different from the other recess 38, so the selection by the surgeon of which recess 36, 38 is used affects the functionality of the surgical retractor. In the preferred embodiment, one of the recesses 36 includes flats 42 (shown in FIGS. 1 and 3-6, 9 and 11, 12 and 14) which project axially relative to the through-hole 40. The other recess 38 does not have such flats, but rather presents a smooth surface 44 surrounding the through-hole 40.

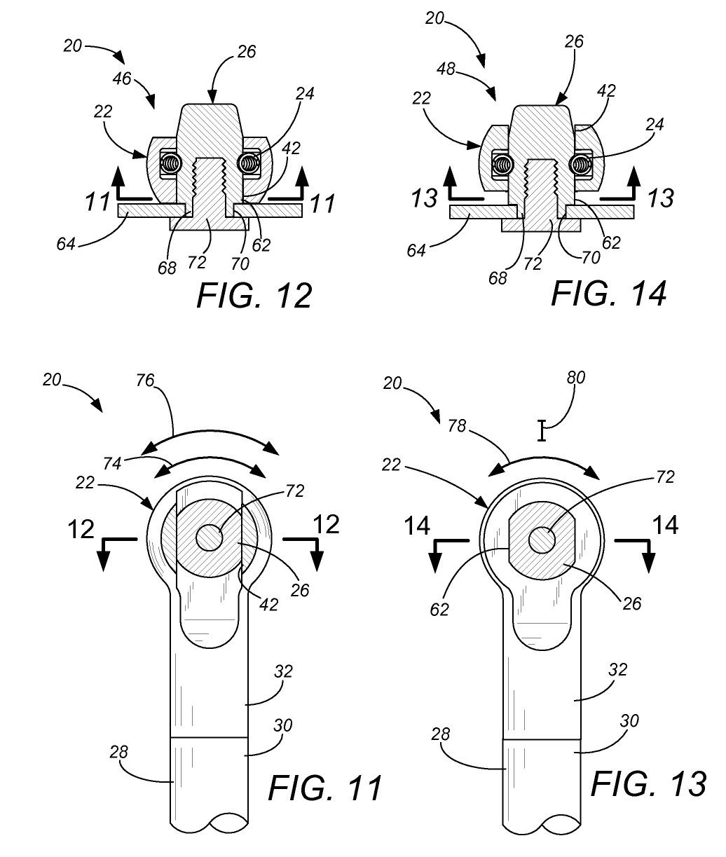

The nipple 26 detachably mates into the through-hole 40 of the receptacle head 22 in either of two directions, i.e., as the receptacle head 22 is depicted in FIG. 1, either from the “bottom” smooth side 46 or from the “top” flatted side 48. FIGS. 11 and 12 depict the nipple 26 received in the flatted side 48 of the receptacle head 22. FIGS. 13 and 14 depict the nipple 26 received in the smooth side 46 of the receptacle head 22.

Formation of the receptacle head 22 is relatively straightforward using methods and materials commonly used in the surgical retractor field. In the preferred embodiment, the receptacle head 22 is machined such as from surgical grade stainless steel. The dimensions of the receptacle head 22 are selected as appropriate for the anticipated load to be encountered during surgical retraction, while providing a low profile to the surgical retraction system. In the preferred embodiment, the through-hole 40 has an overall diameter of about 0.3 inches to receive the nipple 26. To withstand the moment and pull forces which are typically placed on the nipple 26 during retraction, the annular portion 50 of the receptacle head 22 surrounding the through-hole 40 has a radial thickness of about 0.3 inches. The axial thickness of the preferred receptacle head 22, which generally determines the length of the nipple 26 which will be contacted by the receptacle head 22 during retraction, is about ¼ inch, not including the flats 42. The preferred flats 42 extend axially for about another 1/16^{th} of an inch. While the preferred version of the receptacle head 22 shown has a spherical outer profile 52 to provide a sleek appearance and adequate strength, the outer profile could be selected to have virtually any shape. The proximal portion 32 of the receptacle head 22 has a preferred diameter of about ⅜^{th} of an inch for receiving the distal end 30 of the handle portion 28.

The receptacle head 22 includes a detent mechanism 24 for removably receiving the nipple 26. The preferred detent mechanism 24 is a spring having a toroidal shape, which is received during manufacturing assembly of the retractor system in an annular recess 54 positioned at a mid-height of the through-hole 40. The spring 24 mates with an annular recess 56 around the outside of the nipple 26, and sets the desired axial press force for the surgeon to attach the nipple 26 into the receptacle head 22 and the desire axial pull force for the surgeon to remove the nipple 26 from the receptacle head 22. Such toroidal springs 24 are commercially available such as from Bal Seal Engineering Co., Inc. of Foothill Ranch, Calif., commonly referred to as a BAL spring. A different detent mechanism which is well known is a spherical ball (not shown) biased toward the nipple 26 by a compression spring (not shown), which could be used with both the ball and its spring housed in the receptacle head 22.

Formation of the nipple 26 is similarly straightforward using common methods and materials in the surgical retractor field. In the preferred embodiment, the nipple 26 is machined such as from surgical grade stainless steel. The preferred nipple 26 has a cylindrical section 58 of about 0.3 inches in diameter and ¼ inch in height which mates with the through-hole 40 of the receptacle head 22 with just a few mils of radial clearance. A top conical section 60, having a preferred cone angle of about 10-15°, is used to separate the BAL spring 24 during push in. The BAL spring 24 then expands with a tactile click when received in the detent recess 56. The preferred detent recess 56 has a depth of about 0.02 inches and a profile which is simple to machine and mates well with the BAL spring 24, permitting withdrawal of the nipple 26 from the receptacle head 22 with an appropriately strong axially-directed force.

At the base of the cylindrical section 58, the preferred nipple 26 includes flats 62 which are spaced to mate with the flats 42 of the receptacle head 22. The preferred embodiment uses two parallel flats on both the nipple 26 and the receptacle head 22, so there are two mating positions of the nipple flats 62 relative to the receptacle head flats 42 but the shaft 28 always extends perpendicular to the face 66 of the blade 64. Alternatively, the two flats could be at an angle to each other, thereby providing a single mating position of the nipple flats relative to the receptacle head flats. Alternatively, more flats could be provided (such as a hexagon or star pattern), which would allow the surgeon to select the angle of the blade face 66 relative to the support shaft 28.

The preferred nipple 26 also includes structure for subsequent manufacturing attachment onto a retractor blade 64. In the preferred embodiment, the nipple 26 includes an oblong bottom extension 68. An opening 70 is formed in the retractor blade 64, shaped to mate with the oblong bottom extension 68. By having the bottom extension 68 and opening 70 be oblong rather than circular, the nipple 26 cannot spin about its axis relative to the attached retractor blade 64. Once the oblong bottom extension 68 is placed into the opening 70 in the retractor blade 64, a bolt 70 or other fastener can be used to secure the nipple 26 to the retractor blade 64 as known in the art.

FIGS. 10-14 depict how the flip control retractor support works. The surgeon selects whether to perform the attachment of FIGS. 11 and 12 or the attachment of FIGS. 13 and 14. If the surgeon desires to be able to control the angular orientation of the blade 64, the surgeon selects the attachment mode shown in FIGS. 11 and 12. The handle 28 and its attached receptacle head 22 are flipped so the receptacle head flats 42 project downward toward the nipple 26. When the nipple flats 62 are aligned with the receptacle head flats 42 and the retractor blade 64 and nipple 26 are hand pressed into the receptacle head 22, the receptacle head 22 attaches to the nipple 26 with the BAL spring 24 extending into the annular recess of the nipple 26 with a tactile click. In this attachment mode configuration of FIGS. 11 and 12, the surgeon can control the angular orientation of the nipple 26 and attached retractor blade 64 through manipulation of the handle 28. Whichever direction the surgeon positions the handle 28 is the direction that the retractor blade 64 faces. As shown by arrow 74 representing pivoting of the nipple 26 and arrow 76 representing pivoting of the handle 28, the mating flats 42, 62 always cause the nipple 26 and attached retractor blade 64 to face in the direction the handle 28 points, regardless of which direction the surgeon points the handle 28. The surgeon may thus use the retractor in this first “blade secured” mode with the blade 64 being secured relative to the handle 28.

If, however, the surgeon desires to not have the retractor blade 64 consistently face in the direction the handle 28 points, but rather be free to pivot relative to the handle 28, then it is each for the surgeon to change to a second mode of retraction. The surgeon pulls the nipple 26 and attached retractor blade 64 axially out of the flatted recess 36, flips the handle 28 over relative to the blade 64, and reinserts the nipple 26 into the smooth recess 38. The nipple 26 snaps into the recess 38 to be held in place with the detent mechanism 24, and this time the flats 42 on the receptacle head 22 face upward as shown in FIGS. 13 and 14 rather than downward. Now the nipple 26 and retractor blade 64 are free to pivot about the nipple axis as shown by arrow 78, even when the handle 28 is secured in place as shown by line 80. Having the retractor blade 64 free to pivot relative to the handle 28 is beneficial in certain surgical applications, particularly if the direction the blade 64 faces is desired to cause minimal tissue damage during retraction. The surgeon may use the retractor in this second “blade free” mode with the blade 64 free to pivot relative to the handle 28.

In both the “blade secured” and “blade free” modes of tissue retraction, the same BAL spring detent structure 24 can be used to secure the nipple 26/blade 64 relative to the receptacle head 22/handle 28. This reduces the cost as compared to providing two separate recesses each with their own detent mechanism. In both the “blade secured” and “blade free” modes of tissue retraction, the single receptacle head 22 and handle 28 and single blade 64 can be used, again providing greater flexibility for the surgeon with fewer number of parts and lower cost. In both the “blade secured” and “blade free” modes of tissue retraction, the single receptacle head 22 and handle 28 provide the necessary strength for supporting the retraction force while providing a low profile to the retraction system.

While the preferred embodiment involves flipping the handle 28 180° about its longitudinal axis to change between modes of tissue retraction, other embodiments can be envisioned which have more than two modes. For instance, three recesses could be formed into the retractor head 22 for the nipple 26, each offset at 120° circumferential spacing. As another alternative, two through-holes circumferentially 90° from each other could be formed in the receptacle head 22, providing four recesses for receiving the nipple 26. Flats on the receptacle head 22 for the additional nipple-receiving recesses could permit the retractor blade 64 to be fixed at other selected angles (for instance, 15° offset from the direction the handle 28 points, and 300 offset from the direction the handle 28 points). Then the surgeon can quickly select during surgery what angle to place the retractor blade 64 relative to the handle 28. Such “amount of offset” modes of tissue retraction could be provided in two, three, four or any other number of hand detachable recess positions as formed into the receptacle head 22.

Another alternative does not have flats on the retractor head 22 that closely mate with the flats on the nipple 26, but rather permit a desired amount of pivoting play. For instance, one side of the retractor head 22 can secure the nipple 26/blade 64 relative to the handle 28, while the other side of the retractor head 22 permits ±5° of play before the blade 64 is no longer free to pivot relative to the handle 28. Like the “amount of offset” modes of tissue retraction, these “amount of play” modes of tissue retraction could be provided in two, three, four or any other number of hand detachable recess positions as formed into the receptacle head 22.

Another feature involves the breakaway strength of the retractor. In certain surgeries, the surgeon may desire a retraction system which will “break away” if the retraction force reaches a certain threshold, thereby avoiding excessive tissue damage associated with excessive retraction forces during particular events of surgery (such as the patient voluntarily, involuntarily or unconsciously moving his or her body during surgery). Breakaway strength can be established based on various factors of mechanical coupling interaction of the receptacle head 22 and the nipple 26. One such factor is the height of the receptacle head 22 relative to the nipple 26. A simple comparison between FIGS. 12 and 14 shows one set of different heights of the retractor head 22 relative to the nipple 26. In the position of FIG. 12, for instance, the sideways breakaway strength is high, because there is contact between the nipple 26 and the receptacle head 22 throughout the entire height of the receptacle head 22. In the position of FIG. 14, the sideways breakaway strength is lower, since the conical portion of the nipple 26 does not contact the receptacle head 22 and contribute to the breakaway strength, and because the flatted portion of the nipple 26 likewise does not contact the receptacle head 22 and contribute to the breakaway strength. Workers skilled in the art will appreciate that breakaway strength can be easily controlled in both modes based upon the mating shapes of the nipple 26 and the receptacle head 22. Other embodiments change the direction and/or magnitude of the breakaway strength, by changing the dimensions and/or locations of contact surfaces between the receptacle head and nipple.

Additional embodiments can be identified in any situation in which the surgeon wants to be able to quickly change from a first mode of surgical retraction to a second mode of surgical retraction, with both modes provided based upon the mechanical coupling occurring in the receptacle head 22. Various combinations of retraction modes are possible. The surgeon can readily hand-detach the nipple 26/blade 64, flip the handle 28 to the second mode orientation, and reattach the nipple 26/blade 64 to switch between modes of retraction. The additional functionality provided through the multiple modes of retraction use is achieved at low cost, in a flip connection 20 which contains few parts, is easy to manufacture, is easy to sterilize, is strong and robust, and provides a low surgical profile in all modes of use.

Although the present invention has been described with reference to preferred embodiments, workers skilled in the art will recognize that changes may be made in form and detail without departing from the spirit and scope of the invention.

1. A surgical retraction system comprising:
a first component for contacting tissue of a patient; and
a second component for supporting the force of tissue retraction, the second component extending along a longitudinal axis; and
a detachable mechanical coupling between the first component and the second component, the mechanical coupling having a first attachment position available between the first component and the second component which permits a first mode of tissue retraction, the mechanical coupling having a second attachment position available between the first component and the second component which permits a second mode of tissue retraction;
wherein switching between the first mode of tissue retraction and the second mode of tissue retraction requires:
detachment of the first component from the second component;
rotation of the detached second component about its longitudinal axis relative to the first component to a new circumferential position relative to the first component; and
reattachment of the first component to the second component in the new circumferential position.

2. The surgical retraction system of claim 1, wherein the detachable mechanical coupling comprises:
a nipple secured on the first component; and
two or more nipple receiving recesses on the second component.

3. The surgical retraction system of claim 2, wherein at least one nipple receiving recess is a nipple receiving through-hole which can receive the nipple from one side of the nipple-receiving through-hole in the first mode of tissue retraction, and which can further receive the nipple from a flipside of the nipple-receiving through-hole in the second mode of tissue retraction.

4. The surgical retraction system of claim 3, wherein the nipple comprises an annular recess, and wherein the detachable mechanical coupling comprises a detent received in the annular recess, with the detent being receivable in the annular recess both the in first mode of tissue retraction and in the second mode of tissue retraction.

5. The surgical retraction system of claim 1, wherein the new circumferential position of the second mode of tissue retraction is a circumferential rotation of the second component of 180° from the circumferential position of the first mode of tissue retraction.

6. The surgical retraction system of claim 1, wherein the first mode of tissue retraction permits pivoting of the first component about a pivot axis while providing the retraction force, and wherein the second mode of tissue retraction prevents pivoting of the first component about the pivot axis while providing the retraction force.

7. The surgical retraction system of claim 6, wherein the detachable mechanical coupling comprises flats on the first component and the second component which engage in the second mode of tissue retraction to prevent pivoting of the first component about the pivot axis.

8. The surgical retraction system of claim 1, wherein the first mode of tissue retraction has a first breakaway strength while providing the retraction force, and wherein the second mode of tissue retraction has a second breakaway strength while providing the retraction force, the second breakaway strength being different from the first breakaway strength.

9. The surgical retraction system of claim 1, wherein the first component is a retractor blade, and wherein the second component is a retractor handle.

10. The surgical retraction system of claim 9, wherein the retractor handle is adapted to be hand held.

11. The surgical retraction system of claim 9, wherein the retractor handle is adapted to be table-mounted.

12. A surgical retractor comprising:
a retractor blade for contacting tissue of a patient; and
a retractor handle for supporting the force of tissue retraction; and
a detachable mechanical coupling between the retractor blade and the retractor handle;
wherein retractor handle can be flipped between a first mode of retraction coupling with the retractor blade and a second mode of retraction coupling with the retractor blade.

13. The surgical retractor of claim 12, wherein the first mode of coupling permits pivoting of the retractor about a pivot axis while providing the retraction force, and wherein the second mode of coupling prevents pivoting of the retractor about the pivot axis while providing the retraction force.

14. A surgical retractor handle comprising:
a bar extending along a longitudinal axis;
a through-hole in an end of the bar between a first side and a second side;
at least one flat on the first side in an engagement location about the through-hole; and
an absence of flat on the second side in the engagement location relative to the through-hole;
such the surgical retractor handle can be flipped between a first use position having the flat in the engagement location and a second use position not having the flat in the engagement location.

15. The surgical retractor handle of claim 14, further comprising:
a detent which deflectably extends into a central elevation on the through-hole, such that the detent can be engaged in both the first use position and the second use position.
